(19) United States
(12) Patent Application Publication
(10) Pub. No.: US 2010/0159069 A1
(43) Pub. Date: Jun. 24, 2010

(54) METHOD FOR PREPARING A FERMENTED BEVERAGE WITH A LOW ALCOHOL CONTENT

(75) Inventor: Rene Chelle, Grepiac (FR)

Correspondence Address: YOUNG & THOMPSON, 209 Madison Street, Suite 500, Alexandria, VA, 22314, US

(73) Assignee: AB7 Industries, Deyme (FR)

(21) Appl. No.: 11/917,348

(22) PCT Filed: Jun. 14, 2006
(86) PCT No.: PCT/FR06/01335

§ 371 Date: Dec. 13, 2007

(30) Foreign Application Priority Data
Jun. 17, 2005 (FR) 0506141

(51) Int. Cl. C12G 3/08; C12G 3/02; C12H 1/00; C12M 1/12

(52) U.S. Cl. 426/13; 426/15; 426/592; 435/297.1

(57) ABSTRACT

A method for preparing a fermented beverage with low alcohol content, in particular a wine with low alcohol content, involves only biological methods for fermenting natural fruit juice. The method includes essentially the following steps: a) feeding an aerobic culture reactor containing yeasts in multiplication phase with a first fraction of fruit juice; b) feeding an anaerobic fermentation reactor with a medium loaded with yeast obtained at step a) and with a second fraction of fruit juice, to obtain a must; c) filtering the must to separate the yeast and the resulting fermented beverage. In accordance with one important feature, the relative volumes of the first and second fractions of fruit juice are proportional to the desired final degree of alcohol and final proportion of sugar of the fermented beverage. Also described is a device for preparing such a beverage.

The invention relates to the subject of producing fruit juice based beverages, in particular the preparation of an alcoholic beverage derived from grape juice.

It aim is to provide a method for preparing a wine or other beverage with a reduced alcohol content involving only biological methods for fermenting natural fruit juice. Said method includes essentially producing, from a must with an alcoholic strength of preferably 8° to 15°, a beverage with an alcohol level less than at least 4°. The invention also concerns a device for preparing such a beverage.

By definition, wine is the product of the fermentation of grapes where the sugar contained in the juice is converted into alcohol (actually into ethanol) by the action of yeasts. 17 grams of sugar are needed to obtain one degree of alcohol in a litre of wine, known as the Gay-Lussac degree (° GL), which is defined as the percentage of alcohol by volume in the solution. Most wines offered for consumption contain 8° to 15° of alcohol.

It is common knowledge that the campaign against alcoholism is a serious public health issue and that consumers are turning increasingly to products with lower alcohol levels, often misleadingly called “non-alcoholic”, which allow them to quench their thirst, or simply to prolong the pleasure of drinking, without becoming drunk. The success of so-called non-alcoholic beer illustrates this trend. However, non-alcoholic wines which have been offered to consumers have not been received so favourably. The truth is that the taste characteristics of these wines have hardly developed, drastically reducing the pleasure of drinking them. They are produced by fermenting all of the sugar in the fruit juice, then extracting a part of the resulting alcohol using physical methods, such as distillation. Producing them involves adding another stage which increases the cost. Furthermore, it appears that this extraction method simultaneously removes or decreases most of the components responsible for the organoleptic properties of the wine. This goes a long way towards explaining the poor response to these so-called non-alcoholic wines marketed so far.

This invention resolves these problems thanks to a natural method involving only biological methods through culture and fermentation of yeasts which is less onerous and can be accomplished without using expensive dedicated equipment. This method, which is able to operate continuously and is based on fermentation mechanisms identical to those used commonly in the production of wine, can produce a wine or fermented beverage with a low alcohol level having organoleptic properties which appeal to the consumer. A device suitable for use in running the method described here is also proposed and claimed.

Thus, the aim of this invention is to design a biological method for producing a low alcohol wine or, more generally, a low alcohol fermented beverage whose aromatic qualities are retained. Another aim of the invention is to design a method which will permit the final alcohol content to be adjusted when producing a wine or fermented beverage. Yet another aim is to design a method which will permit the final sugar content to be controlled in a low alcohol fermented beverage.

Again, another aim is to design a convenient device to run the method according to the invention, in particular for the continuous production of a low alcohol fermented beverage. A final aim is to prepare a low alcohol beverage which meets the legal definition of a wine.

According to the invention, using a must with an alcohol strength of 8° to 15° for preference, it is possible to obtain a wine with a low alcohol content which can be, as desired, between 4° and 10° of alcohol. Depending on the outcome required, the end product may contain residual sugar or no sugar at all. The wine may be consumed, therefore, as a wine with a low alcohol content, possessing however the main attributes of a wine of strength 10° to 15° obtained in a traditional manner, that is, organoleptic qualities, aromas, etc. It may also be used as a base in the preparation of low alcohol cocktails comprising this low alcohol wine mixed with other natural fruit juices or in the preparation of sparkling drinks.

The term “fermented beverage” is applied to one which is prepared from a fruit juice, at least a part of whose sugar is converted into alcohol. Thus, a fermented beverage can have more or less sugar and more or less alcohol where, unless indicated to the contrary, the final sugar in the beverage is the sugar present initially in the fruit juice and which has not been consumed by the yeasts. Grape juice is the one most commonly used, but all fruit juices can be used to prepare a beverage according to the invention.

A “wine” is defined as a fermented beverage in which the fermentation has been allowed to continue to its natural conclusion during which all (or nearly all) of the sugar initially present in the fruit juice has been converted into alcohol. A wine generally contains little or no sugar.

“Must” is the term given to a fruit juice during or at the end of the alcoholic fermentation process, and before filtering.

The alcohol strength of a wine, denoted by A°, is defined as the degree of alcohol which the wine will have from this juice after complete or almost complete fermentation of the sugars initially present in said juice.

More precisely, the objective of the subject invention is a method for preparing a fermented beverage with low alcohol content from a natural fruit juice comprising essentially the following steps:

a) feeding an aerobic culture reactor containing yeasts in multiplication phase with a first fraction of fruit juice;
b) feeding an anaerobic fermentation reactor with a medium loaded with yeast obtained at step a) and with a second fraction of fruit juice to obtain a must;
c) filtering the must to separate the yeast and the resulting fermented beverage.

Step a) takes place in strongly aerobic conditions, having a continuous supply of oxygen, generally by bubbling air through, and by agitating the medium sufficiently. The yeasts present in the culture reactor are therefore in multiplication phase and rapidly consume the sugar contained in the fruit juice to supply the energy needed for multiplication. The culture conditions for the yeasts, in particular oenological yeasts, are well known to one versed in the art who knows how to adapt them in relation to the characteristics of the strains used and the nutritional medium.

Step b) works under anaerobic conditions, required for the alcohol fermentation process. The fraction of fruit juice added to the fermentation reactor provides the sugar which is converted into alcohol by the yeasts obtained from the culture reactor. Since the sugar content in the fraction of fruit juice emerging from the culture reactor has been drastically reduced or consumed completely, the amount of sugar fed into the fermentation reactor by the total fruit juice (sum of the two fractions of juice) is reduced accordingly. Thus it is possible to control the amount of sugar fed into the fermentation and, therefore, the degree of alcohol which the wine will have finally when emerging from the method according to the invention. To do this, all that is required is to calculate, knowing the initial sugar content in the juice, what volume of juice must be fed into each of the reactors to have the desired amount of sugar in the fermenter to convert into alcohol.

Thus, according to one important feature of the invention, the relative volumes of the first and second fractions of fruit juice are proportional to the desired final alcoholic degree of the fermented beverage.

It is also possible to produce a wine or a naturally sugared beverage by ensuring that only a part of the sugar is consumed in the fermenter. This can be obtained in different ways, such as by lessening the time the must stays in the fermenter. In this instance, the respective volumes of the fruit juice fractions fed into each of the reactors are arranged according to the desired residual quantity of sugar.

According to another beneficial feature of the invention, the respective volumes of the first and second fruit juice fractions are proportional to the final alcoholic degree and to the final sugar level desired in the fermented beverage.

According to a preferred version of the invention, the sugar in the first juice fraction is completely or nearly completely consumed in the culture reactor. This then produces, at the outlet of the culture reactor, a medium containing yeast cells, the liquid phase of which comprises essentially fruit juice from which its sugar has been removed. This phase therefore performs the dual functions of producing large quantities of yeast and reducing the quantity of sugar of the total fruit juice which is then fed into the fermentation.

When the method is being started, the first step is simply to initiate the cell culture in the culture reactor, then, when the concentration is sufficient to provide a steady supply of yeast to the fermentation reactor while, at the same time, maintaining a high multiplication level, the fermentation is started. The yeasts present in the fermentation reactor originate mainly, therefore, from the culture reactor. Their concentration must be relatively high to provide rapid fermentation once transferred into the fermentation reactor with the second juice fraction. According to a characteristic preferred in the inventive method, the level b) is attained when a yeast concentration of at least 60 gm/l is reached, and preferably at least 90 gm/l, in the culture reactor.

According to another way of working the invention, rather than feeding the second fraction of the fruit juice directly into the fermenter it is possible to feed it together with the first fraction into the culture reactor. In effect, the two juice fractions are mixed, but the volume of each of these theoretical fractions has been calculated according to the principle described above such that it is possible to attribute to each fraction the quantity of sugar corresponding to it and to think in terms of the equivalent quantities of sugar, even though, as has been stated already, they are not physically separated. According to the invention, the juice has to stay in this reactor just long enough for a quantity of sugar equivalent to the theoretical sugar in the first fraction to be consumed and allowing the same cell multiplication to occur as described in the alternative operation of the invention described above. The medium thus obtained, charged with yeasts and containing sugar equivalent to that of the second fraction, is then fed in to the fermentation reactor. Everything then proceeds as if the two fractions had been separated.

Thus, according to one version of carrying out the invention, at step a), the first and second fractions of the fruit juice are fed into the culture reactor, then they are fed together into the fermentation reactor.

In this version, the quantity of juice fed into the culture reactor is larger and the concentration of yeast is reduced as a result. This is why, preferably, step b) is performed when the equivalent quantity of sugar in the first fraction is consumed completely or almost completely in the culture reactor and when a concentration of yeasts of at least 30 gm/l, and preferably at least 45 gm/l, has been reached in the culture reactor.

The yeasts in the multiplication phase in the culture reactor can, of course, be the yeasts naturally present in the fruit juice or inoculated exogenous yeasts. It is a known fact that the juices contain endogenous flora consisting of bacteria and yeasts. The endogenous yeasts may be present, therefore, at the start of the culture or it is even possible to initiate the culture using a starter consisting of a micro-organism preparation.

When inoculating yeasts selected for their fermenting properties, or for organoleptic qualities, it is preferable that other strains do not grow. To prevent this, the fruit juice can be sterilised or treated in some other way before using it in the inventive method. This is why, in an alternative method, the exogenous yeasts are cultivated in a fruit juice treated beforehand to destroy all or part of its endogenous flora.

According to another preferred version of the method according to the invention, a supplementary supply of nutrients can be provided in the culture reactor. This supply is calculated so that the yeast population, multiplying naturally, consumes the main part of this nutrient supply, selected by one versed in the art from among the traditional substances available to him

Barring any hitch specifically inherent in the microbiological processes which an expert in the field would know how to resolve, the microbic populations involved in the vinification (mainly the yeast populations) will thus be available always in quantities capable of producing the alcohol without undesirable strains being able to colonise the must.

In view of the large concentration of available yeasts and the reduction in final alcohol content, it is possible to achieve far higher vinification speeds than normal, considerably reducing the time spent by the must in the fermentation reactor, increasing the productivity of the reactor accordingly. Based on this abnormal performance, it is possible to consider working at much lower temperatures despite their moderating effect on the metabolism, in order to conserve the aromas, particularly the volatile aromas. A temperature of about 8° to 15° is possible. The subject method which speeds up the processing while modifying the characteristics of the end product therefore also has the advantage of not introducing a parasitic taste.

To increase the concentration of yeasts in the fermentation reactor even more, and, thus, the performance of the method according to the invention, it would be possible, if needed, to recycle all or some of the yeasts recovered by the must filtration system and feed them again into the fermenter or even into the culture reactor without altering the taste of the wine. Thus, according to one preferred characteristic of the invention, at least a part of the yeasts collected after filtration is fed once more into the fermentation reactor or into the culture reactor.

According to another preferred characteristic of the invention, at step c) the must is filtered using a filtering method selected from frontal filtration, centrifugation or tangential ultrafiltration. Where tangential ultrafiltration is used, the end product obtained is practically sterile. Furthermore, this method makes it possible to maintain a certain pressure in order to obtain a fizzy drink or even a sparkling one.

In a preferred processing method, the operating conditions are arranged so as to impart to the end product, and in particular in the case of producing wine, the aromatic characteristics of a good wine despite the low alcohol content which, according to general opinion, is considered to be a handicap from this point of view. These operating conditions are arranged to provide the aromas obtained when the alcohol level is above 5° GL, that is, in the final phase when the sugar is consumed. In particular, the must obtained emerging from the fermenter can undergo several other treatments, known as maturation, before being filtered.

According to one interesting characteristic, the must rests in a maturation reactor before the filtration step c). Maturation comprises a treatment intended to enrich the must aromas, such as promoting accelerated aging when legislation allows it, or again trapping the volatile aromatic products produced during fermentation. The two processes can be carried out beneficially together and done in the same reactor.

According to a preferred mode of operation, the must passes through in the maturation reactor, percolating through packed wood chips. The must can be fed, for example, at a high point in the maturation reactor and allowed to flow through the wood chips. This treatment, allowed under the law in certain countries, is designed to improve the taste qualities of the end product by promoting accelerated aging.

According to another preferred mode of operation, the gaseous mixture charged with volatile aromas emerging from the fermentation reactor passes through the must resting in the maturation reactor. Again, for preference, the gaseous mixture charged with volatile aromas leaving the culture reactor passes through the must resting in the maturation reactor.

For example, the maturation reactor can receive, at a low point, the gaseous mixture leaving the culture reactor and the gas emerging from the fermentation reactor, carrying with them the volatile molecules of the light aromas. The gas passes through, rising as it does, against the flow of the must as it matures, the latter trapping these small molecules which enriches its organoleptic profile.

This mechanism is more effective at low temperatures. This is why, for preference, the said maturation reactor is kept at a temperature between 2° C. and 10° C. by refrigeration which aids the trapping method. An outlet is provided in its upper region.

According to a beneficial characteristic of the invention, the substance transfers take place continuously at a speed controlled by valves. In fact, depending on the desired levels of sugar and alcohol in the fermented product produced by the method according to the invention, as well as the initial quantities of fruit juice fed into the reactors, the kinetic characteristics play an important role. The time during which the fruit juice or the must remains in the different reactors also becomes a parameter that needs to be monitored to obtain a sufficient speed for the reactions. In a continuous method, the feeding speed and the transfer of substances from one reactor to the other, including towards the filtration system, have to be controlled. Valves are therefore placed judiciously to provide control and regulation of the flow of material.

The end product obtained by the method described above is a wine or fermented beverage, ready for consumption, or capable of having supplementary treatment. For example, a malo-lactic fermentation stage can be arranged. The product could be marketed as a low alcohol wine-type beverage but having the characteristics of a good quality wine. Optionally, it could have the juice of other fruits added to make cocktails. These latter fruit juices could also contain small amounts of alcohol from fermentation of their sugar by the method according to the subject invention. Beverages thus obtained will contain alcohol and sugar at levels which depend on the conditions selected for the method.

The invention also concerns a device for preparing a fermented beverage with low alcohol content from a natural fruit juice, comprising:
a reactor suitable for carrying out the aerobic culture of yeasts in multiplication phase,
a reactor suitable for carrying out the anaerobic fermentation of a fruit juice by the yeasts, each culture and fermentation reactor having the means to feed a defined fraction of fruit juice,
a unit to filter the fermented product, and
means for transferring material from the culture reactor to the fermentation reactor, and from the fermentation reactor to the filtration unit.

The different elements of this device are all designed specifically to carry out the method just described due to their specific layout. This means that the device according to the invention can be described succinctly with the understanding that the operating conditions as defined for the said method will show to one versed in the art the way the elements in the device are constructed. In particular, the culture reactors and the fermentation reactors, the filtration systems are well known items of equipment in themselves and need not be described in detail.

According to one feature of the device according to the invention, the culture reactor has a means of aeration, a mixer and means of regulating the temperature. It provides for the growth of the yeasts under highly aerobic conditions to support active cellular multiplication.

According to another feature of the device according to the invention, the fermentation reactor has a means of aeration, a mixer and means of regulating the temperature. It is suitable for operating under anaerobic conditions needed for the alcohol fermentation process.

These two reactors are fitted with means of regulating the temperature to provide control of the reaction speeds in accordance with the defined production objective and to retain the aromas more effectively. More generally, the device according to the invention has means of controlling the working parameters such as the flows, temperatures, the pH, etc, which an expert in the art would know how to select and operate.

According to another feature of the device according to the invention, the filtration unit is selected from either a frontal filter, a centrifuge or a tangential ultrafilter. A tangential ultrafiltration unit produces an end product that is practically sterile. Furthermore, it is able to control the carbon dioxide content.

In addition, according to a particular method of constructing the device according to the invention, the device has at least one maturation reactor upstream of the filtration unit to confer to the end product, and notably in the case where a wine is being produced, the aromatic characteristics of a good quality wine, despite the low alcohol content.

The maturation reactor may be equipped in particular with one of more of the following elements:
packed wood chips,
an inlet at its base for the gas mixture charged with the volatile aromas from the fermentation reactor,
an inlet at its base for the gas mixture from the culture reactor,
means of regulating the temperature.

The respective function and use of these elements for the quality of the end product have been explained in detail above. Preferably, the maturation reactor has gas entering at a low point from the culture reactor and gas entering at a low point from the fermentation reactor. The gas flows entering the maturation reactor from the culture and fermentation reactors pass through the must, circulating upwards and exchanging their aromas with the liquid phase. An outlet is provided at the top of the maturation reactor.

According to a particular method of arranging the device according to the invention, the filtration unit is fitted with means of transferring the material retained by the filter to the fermentation reactor. It can consist, for example, of a single pipe fitted with a pump.

It should be noted that the fermentation process can continue, as long as sugar still remains to be consumed, up to the separation of the yeasts from the wine.

Regardless of the means of operation selected, when the device according to the invention is operating in continuous mode, it preferably has means of regulating the speed at which material is transferred to each reactor, in particular valves or solenoid valves.

The method and the device according to the invention described here are designed especially to be used for the preparation of a fermented beverage with low alcohol content from a natural fruit juice. This preparation is made from the components naturally present in the materials initially used, which can be grape juice, the juice of another fruit, or a mixture of both.

A claim is made also for a fermented beverage with low alcohol content obtained from a natural fruit juice by the method according to the invention. The sugar content of this beverage can vary and can be still, with a degree of controlled turbulence.

Other benefits and properties of interest will become clearer using the following examples as illustrations.

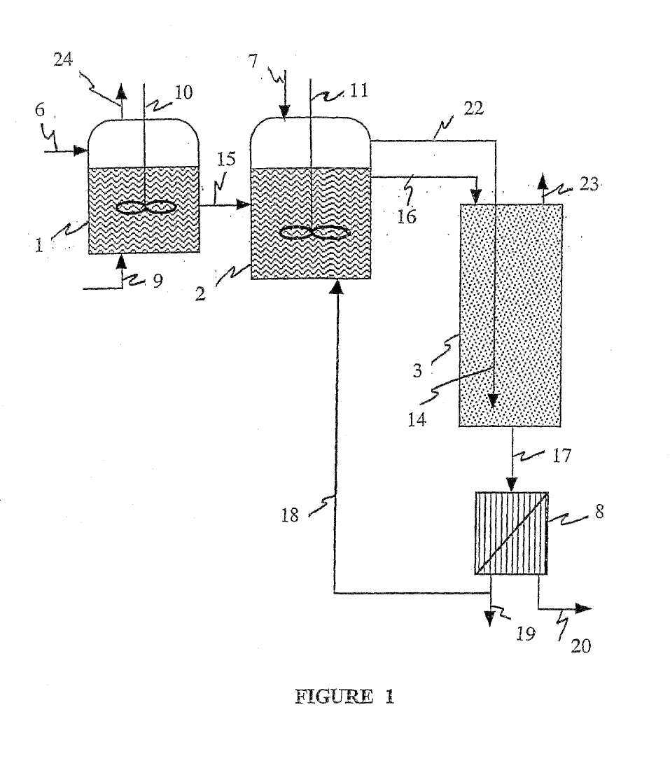

FIG. 1 represents a diagram of a device according to the invention, comprising a culture reactor and a fermentation reactor, a filtration system and a maturation reactor to retain aromas.

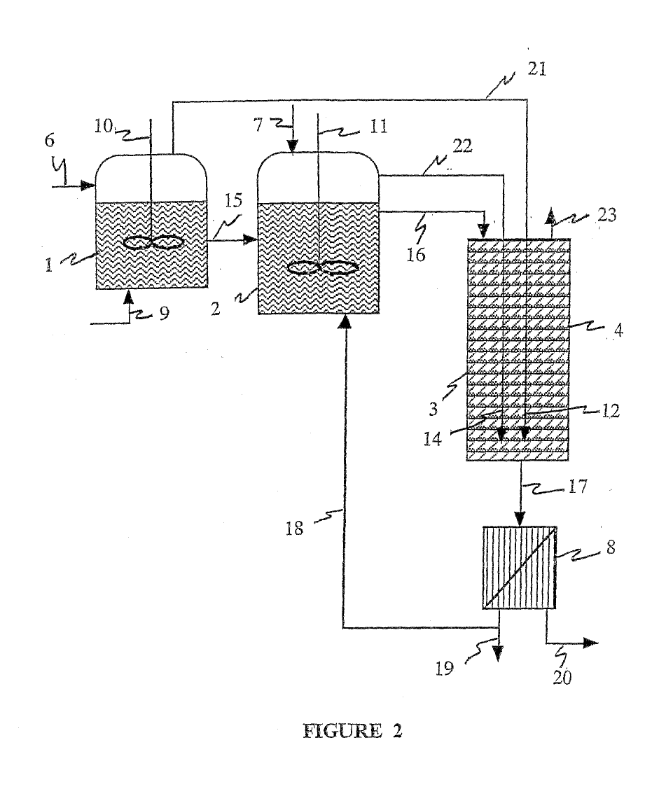

FIG. 2 represents a diagram of a device according to the invention, comprising a culture reactor and a fermentation reactor, a filtration system and a maturation reactor for accelerated aging and to retain aromas.

### EXAMPLE 1

The method according to the invention can be carried out using the device represented in FIG. 1. This device comprises the culture reactor 1, the fermentation reactor 2, a filtration system 8 and the maturation reactor 3.

The culture reactor is designed to carry out the aerobic culture of yeasts in the multiplication phase. It is fed by the first fraction of the fruit juice via the pipe at inlet 6. It is fitted with an air feed 9 and mixer 10 to ensure good oxygenising of the culture medium.

The fermentation reactor 2 is designed to carry out the anaerobic fermentation of the second fraction of the fruit juice fed in via pipe 7, using the yeasts arriving from the culture reactor 1 via the connecting pipe 15. It is fitted with a mixer 11 to homogenise the medium. The must is transferred from the fermentation reactor 2 to the maturation reactor 3 via the pipe 16, then to the tangential filter 8 via the pipe 17.

The maturation reactor 3 has a low inlet 14 for the gas coming from the fermentation reactor 2 via the pipe 22. It also has an outlet 23.

The filtration unit 8 is equipped with pipe 18 for transferring a part of the yeasts retained by the filter to the fermentation reactor 2. The pipe 19 ensures that excess yeasts are removed.

The transfer of material to each reactor and unit is controlled by valves (not shown), located for example in the inlet and/or outlet pipes 6, 7, 15, 16, 17, 18, 22.

Pipe 20 gathers the end product, wine or fermented beverage according to the operating conditions selected.

### EXAMPLE 2

The method according to the invention can be carried out also using the device represented in FIG. 2. This device comprises the culture reactor 1, the fermentation reactor 2, a filtration system 8 and the maturation reactor 3 for accelerated aging and to retain aromas.

The culture reactor 1 and the fermentation reactor 2 are identical to those described in FIG. 1. The must is transferred from the fermentation reactor 2 to the maturation reactor 3 via the pipe 16, then to the tangential filter 8 via the pipe 17.

The maturation reactor 3 has a low inlet 12 for the gas coming from the culture reactor 1 via the pipe 21 and a low inlet 14 for the gas coming from the fermentation reactor 2 via the pipe 22. It also has an outlet 23. It contains a wood chip pack 4 through which the must percolates. It is refrigerated.

The transfer of material to each reactor and unit is controlled by valves (not shown), located for example in the inlet and/or outlet pipes 6, 7, 15, 16, 17, 18, 21 and 22.

Pipe 20 gathers the end product, sparkling wine or fizzy sugared beverage according to the operating conditions selected.

### EXAMPLE 3

Wine could be produced, containing no sugar and with an alcohol level denoted as D° from a volume V of grape juice with a potential alcohol content denoted by A°, by using the device in Example 1. The degree of alcohol in the wine produced will be given by D°=yA°, where 0<y<1. The method takes place in continuous mode.

Two fractions are prepared from volume V of grape juice such that the volume of the first fraction F₁ is V₁=xV and the volume of the second fraction F₂ is V₂=yV, where x+y=1.

Firstly, the culture reactor 1 is loaded with yeasts, the strain of which is a function of the oenological and aromatic characteristics of the beverage it is desired to produce. The nutrients, in particular minerals, needed for cellular multiplication are also added.

Then the fraction F₁ of the juice is fed continuously into the culture reactor 1, agitated vigorously by the mixer 10 and aerated. The yeasts are produced at the optimal temperature T₁ to provide the best growth conditions, thus minimising the time spent in this reactor 1. All of the sugar introduced by the volume V₁ of the fraction F₁ is assimilated by the yeasts producing 45 gm of yeasts for each 100 gm of sugar introduced.

The culture medium containing the yeasts is drawn off continuously by pipe 15 and transferred to the fermentation reactor 2. This reactor is fed in parallel via the pipe 7 with fraction F₂ of the juice. The must thus formed is agitated by the mixer 11 (not absolutely necessary) and operates anaerobically at temperature T₂ selected to obtain the best organoleptic characteristics in the wine produced. The must is then transferred to the maturation reactor 3. It enters at the top and continues to flow, all the time in contact with the gaseous mixture entering from the culture reactor 1 via the pipe 21, and with the gas entering from the fermentation reactor 2 via the pipe 22, where the gas has been fed in at a low point in the reactor 3, leaving via the vent 23. The maturation reactor 3 is refrigerated at a temperature T₃ of 2° to 10° C. to improve entrapment of the aromas.

The quantity of sugar contained in volume V₂ is then converted totally into alcohol.

In another way of operating the method, the gas escaping from culture reactor 1 can be released into the atmosphere via vent 24 or even bubbled or percolated through the must in a maturation reactor to retain the aromas.

The wine is drawn off continuously through the pipe 20. Its alcohol content is given by D°=yA° since only the sugar contained in volume yV has been subjected to fermentation and the alcohol produced is in the final volume of the wine obtained which is close to the initial volume of fruit juice fed into the device, that is, V=V1+V2.

To produce a sugared beverage, the same principle is applied, but the calculation of the volumes V1+V2 must take account of the desired quantity of sugar to remain in the beverage.

1. A method for preparing a fermented beverage with low alcohol content from a natural fruit juice, characterised in that said method includes essentially the following steps comprising:
a)—feeding an aerobic culture reactor (1) containing yeasts in multiplication phase with a first fraction of fruit juice;
b)—feeding an anaerobic fermentation reactor (2) with a medium loaded with yeast obtained at step a) and with a second fraction of fruit juice to obtain a must;
c)—filtering the must to separate the yeast and the resulting fermented beverage.

2. A method in accordance with claim 1, characterised in that the relative volumes of the first and second fractions of fruit juice are proportional to the desired final degree of alcohol of the fermented beverage.

3. A method in accordance with claim 1, characterised in that the relative volumes of the first and second fractions of fruit juice are proportional to the desired final degree of alcohol and the final proportion of sugar of the fermented beverage.

4. A method in accordance with claim 1, characterised in that the sugar in the first juice fraction is completely or nearly completely consumed in the culture reactor (1).

5. A method in accordance with claim 1, characterised in that the level b) is attained when a yeast concentration of at least 60 gm/l is reached, and preferably at least 90 gm/l, in the culture reactor (1).

6. A method in accordance with claim 1, characterised in that the first and second fractions of fruit juice are fed into the culture reactor (1), then both are fed together into the fermentation reactor (2).

7. A method in accordance with claim 6, characterised in that step b) is performed when a concentration of yeasts of at least 30 gm/l, and preferably at least 45 gm/l, has been reached in the culture reactor (1) and when the equivalent quantity of sugar in the first fraction is consumed completely or almost completely in the culture reactor (1).

8. A method in accordance with claim 1, characterised in that the yeasts in the multiplication phase in the culture reactor (1) can be the yeasts naturally present in the fruit juice or inoculated exogenous yeasts.

9. A method in accordance with claim 1, characterised in that the exogenous yeasts are cultivated in a fruit juice treated beforehand to destroy all or part of its endogenous flora.

10. A method in accordance with claim 1, characterised in that a supplementary supply of nutrients can be provided in the culture reactor (1).

11. A method in accordance with claim 1, characterised in that the must rests in a maturation reactor (3) before the filtration step c).

12. A method in accordance with claim 8, characterised in that the must passes through in the maturation reactor, percolating through packed wood chips (4).

13. A method in accordance with claim 12, characterised in that the gaseous mixture charged with volatile aromas emerging from the fermentation reactor (2) passes through the must resting in the maturation reactor (3).

14. A method in accordance with claim 13, characterised in that the gaseous mixture charged with volatile aromas emerging from the culture reactor (1) passes through the must resting in the maturation reactor (3).

15. A method in accordance with claim 14, characterised in that the said maturation reactor (3) is kept at a temperature between 2° C. and 10° C.

16. A method in accordance with claim 1, characterised in that, at step c), the must is filtered using a filtering method selected from frontal filtration, centrifugation or tangential ultrafiltration.

17. A method in accordance with claim 1, characterised in that at least a part of the yeasts collected after filtration is fed once more into the fermentation reactor (2) or into the culture reactor (1).

18. A method in accordance with claim 1, characterised in that the transfers of material take place in continuous mode at a speed which can be controlled.

19. A device for preparing a fermented beverage with low alcohol content from a natural fruit juice, characterised in that it comprises:
a reactor (1) suitable for carrying out the aerobic culture of yeasts in multiplication phase,
a reactor (2) suitable for carrying out the anaerobic fermentation of a fruit juice by the yeasts, each culture and fermentation reactor having the means (6, 7) to feed a defined fraction of fruit juice,
a unit (8) to filter the fermented product, and
means for transferring material from the culture reactor (1) to the fermentation reactor (2), and from the fermentation reactor (2) to the filtration unit (8).

20. A device in accordance with claim 19, characterised in that the culture reactor (1) has a means of aeration (9), a mixer (10) and means of regulating the temperature.

21. A device in accordance with claim 19, characterised in that the fermentation reactor (2) has a mixer (11) and means of regulating the temperature.

22. A device in accordance with claim 19, characterised in that the filtration unit (8) is selected from either a frontal filter, a centrifuge or a tangential ultrafilter.

23. A device in accordance with claim 19, characterised in that it also has at least one maturation reactor (3) upstream of the filtration unit (8).

24. A device in accordance with claim 23, characterised in that the maturation reactor (3) may be equipped with one of more of the following elements:
packed wood chips (4),
an inlet (14) at its base for the gas mixture charged with the volatile aromas from the fermentation reactor (2),
an inlet (12) at its base for the gas mixture from the culture reactor (1),
means of regulating the temperature.

25. A device in accordance with claim 19, characterised in that the filtration unit (8) is fitted with means of transferring the material retained by the filter to the fermentation reactor (2).

26. A device in accordance with claim 19, characterised in that it has means of regulating the speed at which material is transferred to each reactor.

27. (canceled)

28. A fermented beverage with low alcohol content obtained from a natural fruit juice by a method in accordance with claim 1.
